(19) United States
(12) Patent Application Publication
(10) Pub. No.: US 2018/0271539 A1
(43) Pub. Date: Sep. 27, 2018

(54) DEVICES, SYSTEMS, AND METHODS FOR TREATING ANEURYSMS AND OTHER MEDICAL CONDITIONS

(71) Applicant: Ghassan S. Kassab, La Jolla, CA (US)

(72) Inventor: Ghassan S. Kassab, La Jolla, CA (US)

(21) Appl. No.: 15/926,291

(22) Filed: Mar. 20, 2018

Related U.S. Application Data
(60) Provisional application No. 62/518,643, filed on Jun. 13, 2017.
(60) Provisional application No. 62/473,724, filed on Mar. 20, 2017.

(51) Int. Cl. A61B 17/12; A61M 31/00; A61N 5/06; A61L 31/14; A61L 31/00

(52) U.S. Cl. CPC A61B 17/12136; A61M 31/002; A61B 17/1204; A61B 17/12113; A61B 2017/00951; A61L 31/14; A61L 31/005; A61L 31/148; A61N 5/062

(57) ABSTRACT

Devices, systems, and methods for treating aneurysms and other medical conditions. A method of the present disclosure includes the steps of introducing a device into a lumen of a mammalian luminal organ at or near an aneurysm, the device comprising an expandable member, and a material layer coupled to the expandable member, wherein the expandable member is configured to attach to an elongated body configured to deliver an inflation material therethrough and into the expandable member, and inflating the expandable member so that the material layer contacts the mammalian luminal organ and adheres to the mammalian luminal organ at or near the aneurysm.

### PRIORITY

The present application is related to, and claims the priority benefit of, a) U.S. Provisional Patent Application Ser. No. 62/518,643, filed Jun. 13, 2017, and b) U.S. Provisional Patent Application Ser. No. 62/473,724, filed Mar. 20, 2017, the entire contents of which are incorporated herein directly and by reference in their entirety.

### BACKGROUND

The current treatment of aneurysms generally includes the use of a device having some sort of stent frame that is ultimately delivered into the blood vessel having the aneurysm and expanded therein. However, said procedures, while having some successes in connection with aneurysm treatment, are now known have significant side effects, including hypertension.

In view of the foregoing, devices and systems to treat aneurysms, and methods of using the same to treat aneurysms, without said side effects would be well received in the medical arts.

### BRIEF SUMMARY

The present disclosure includes disclosure of devices and systems to treat aneurysms and methods of using the same to treat aneurysms. Various devices of the present disclosure comprise a balloon and use a material layer to treat aneurysms, such as cerebral aneurysms or aortic aneurysms, without the use of stents, coils (such as for cerebral aneurysms) or endografts or endoprostheses (such as for aortic aneurysms).

The present disclosure includes disclosure of a device, comprising an expandable member, and a material layer coupled to the expandable member, wherein the expandable member is configured to attach to an elongated body configured to deliver an inflation material therethrough and into the expandable member.

The present disclosure includes disclosure of a device, wherein the material layer is pre-adhered to the expandable member using an adhesive.

The present disclosure includes disclosure of a device, wherein the material layer does not completely encircle a portion of the expandable member.

The present disclosure includes disclosure of a device, wherein the expandable member comprises a mammalian tissue selected from the group consisting of pulmonary ligament, parietal pleura, mediastinal pleura, and visceral pleura.

The present disclosure includes disclosure of a device, wherein the expandable member comprises a non-mammalian tissue.

The present disclosure includes disclosure of a device, wherein the expandable member is not configured as a stent, a coil, an endograft, or an endoprosthesis.

The present disclosure includes disclosure of a device, wherein the material layer is configured as a patch.

The present disclosure includes disclosure of a device, wherein the material layer is configured as a cylinder.

The present disclosure includes disclosure of a device, further comprising an ultraviolet light sensitive adhesive positioned on a relative outside of the material layer, the ultraviolet light sensitive adhesive configured to cure or otherwise activate from ultraviolet light so to cause the ultraviolet light sensitive adhesive to adhere the material layer to a mammalian luminal organ when the device is positioned within a lumen of the mammalian luminal organ and when the expandable member is expanded.

The present disclosure includes disclosure of a device, further comprising an ultraviolet light sensitive adhesive positioned on a relative outside of the material layer, the ultraviolet light sensitive adhesive configured to cure or otherwise activate from a first wavelength of ultraviolet light so to cause the ultraviolet light sensitive adhesive to adhere the material layer to a mammalian luminal organ when the device is positioned within a lumen of the mammalian luminal organ and when the expandable member is expanded, and wherein the ultraviolet light sensitive adhesive is further configured to deactivate from a second wavelength of ultraviolet light so to cause the ultraviolet light sensitive adhesive to detach the material layer from the mammalian luminal organ.

The present disclosure includes disclosure of a device, further comprising one or more light sources positioned within the expandable member.

The present disclosure includes disclosure of a device, further comprising one or more light sources positioned upon an outer surface of the expandable member.

The present disclosure includes disclosure of a device, further comprising a drug release portion positioned upon the material layer, so that when the expandable member is inflated within a lumen of a mammalian luminal organ, the drug release portion contact the mammalian luminal organ to allow drug from the drug release portion to be delivered to the mammalian luminal organ.

The present disclosure includes disclosure of a system, comprising a device, comprising an expandable member and a material layer coupled to the expandable member; an elongated body coupled to the expandable member; and a chemical delivery source configured to couple to the elongated body; wherein the elongated body is configured to deliver an inflation material from the chemical delivery source therethrough and into the expandable member.

The present disclosure includes disclosure of a system, further comprising a light device configured for insertion into a lumen of a mammalian luminal organ with the device, the light device further configured to generate ultraviolet light within the lumen.

The present disclosure includes disclosure of a method, comprising the steps of introducing a device into a lumen of a mammalian luminal organ at or near an aneurysm, the device comprising an expandable member; and a material layer coupled to the expandable member; wherein the expandable member is configured to attach to an elongated body configured to deliver an inflation material therethrough and into the expandable member; and inflating the expandable member so that the material layer contacts the mammalian luminal organ and adheres to the mammalian luminal organ at or near the aneurysm.

The present disclosure includes disclosure of a method, further comprising the step of deflating the expandable member so to cause the expandable member to detach from the material layer.

The present disclosure includes disclosure of a method, wherein the material layer has an adhesive positioned thereon, and wherein the step of inflating is performed to cause the adhesive of the material layer to contact the mammalian luminal organ and to cause the material layer to adhere to the mammalian luminal organ.

The present disclosure includes disclosure of a method, further comprising the step of introducing a chemical into the expandable member to cause an adhesive applied to the expandable member to weaken to allow the expandable member to disengage from the material layer.

The present disclosure includes disclosure of a method, wherein the step of inflating is performed to cause the material layer to adhere to the mammalian luminal organ and to completely cover the aneurysm.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a side view of a device comprising a balloon having a material layer configured as a patch thereupon, according to an exemplary embodiment of the present disclosure;

FIG. 2 shows a side perspective view of a device comprising a balloon having a material layer configured as a cylinder thereupon, according to an exemplary embodiment of the present disclosure;

FIG. 3 shows a material layer configured as a patch having a length and a width, according to an exemplary embodiment of the present disclosure;

FIG. 4 shows a material layer configured as a patch having a diameter, according to an exemplary embodiment of the present disclosure;

FIG. 5 shows a material layer configured as a cylinder having a length and a diameter, according to an exemplary embodiment of the present disclosure;

FIG. 6 shows a device comprising a balloon having a material layer configured as a patch thereupon, positioned within a lumen of mammalian luminal organ, wherein the balloon is in a fully or partially deflated state, according to an exemplary embodiment of the present disclosure;

FIG. 7 shows a device comprising a balloon having a material layer configured as a patch thereupon, positioned within a lumen of mammalian luminal organ, wherein the balloon is in a fully or partially inflated state, according to an exemplary embodiment of the present disclosure;

FIG. 8 shows a balloon having a balloon adhesive applied thereto, according to an exemplary embodiment of the present disclosure;

FIG. 9 shows a device comprising a balloon with a catheter coupled thereto and with the catheter operably connected to an inflation source, according to an exemplary embodiment of the present disclosure;

FIG. 10 shows a cross-sectional view of a catheter having a first lumen, according to an exemplary embodiment of the present disclosure;

FIG. 11 shows a cross-sectional view of a catheter having a first lumen and a second lumen, according to an exemplary embodiment of the present disclosure;

FIG. 12 shows a side view of a light device comprising a light source, according to an exemplary embodiment of the present disclosure;

FIG. 13 shows a chemical delivery source with a catheter coupled thereto, according to an exemplary embodiment of the present disclosure;

FIG. 14 shows a block component diagram of components of an exemplary system of the present disclosure, according to an exemplary embodiment of the present disclosure; and

FIG. 15 shows an embodiment of a device, according to an exemplary embodiment of the present disclosure.

An overview of the features, functions and/or configurations of the components depicted in the various figures will now be presented. It should be appreciated that not all of the features of the components of the figures are necessarily described. Some of these non-discussed features, such as various couplers, etc., as well as discussed features are inherent from the figures themselves. Other non-discussed features may be inherent in component geometry and/or configuration.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The present disclosure includes disclosure of devices and systems to treat aneurysms and methods of using the same to treat aneurysms.

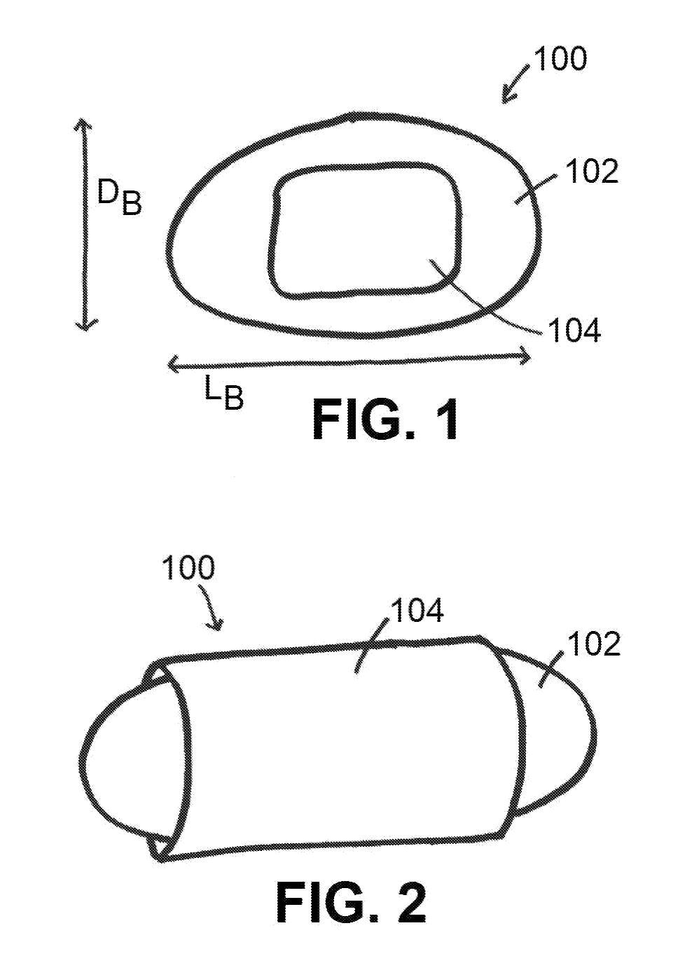

An exemplary device for treating aneurysms of the present disclosure is shown in FIG. 1. As shown in FIG. 1, an exemplary device 100 comprises an expandable member (referred to as a balloon 102 herein), configured for expansion within a mammalian luminal organ. Balloons 102 of the present disclosure are further configured to receive a material layer 104 positioned thereon, such as being pre-adhered to balloon 102. Material layers 104 of the present disclosure can comprise patches, such as a material layer 104 that does not completely encircle a portion of balloon 102 (such as shown in FIG. 1), or can comprise cylinders, such as a material layer 104 that completely encircles a portion of balloon 102 (such as shown in FIG. 2).

Exemplary material layers 104 of the present disclosure can comprise any number of biologically-compatible materials, including, but not limited to, mammalian tissues, such as, for example, lung (pulmonary) ligament, parietal pleura, mediastinal pleura, visceral pleura, and other mammalian tissues. As referenced herein, pulmonary ligament and visceral pleura are both “pulmonary region” tissue. Alternatively, mediastinal pleura is parietal pleura, which is the portion of the pleura external to the pulmonary pleura. Material layers 104 may also comprise non-mammalian tissues, such as various biologically-compatible materials used in the medical arts in connection with patient treatment. Material layers 104 of the present disclosure, however, would not comprise stents, coils, endografts, endoprostheses, etc., as material layers 104 of the present disclosure do not require reinforcement that would generally be provided by said stents, coils, endografts, endoprostheses, etc.

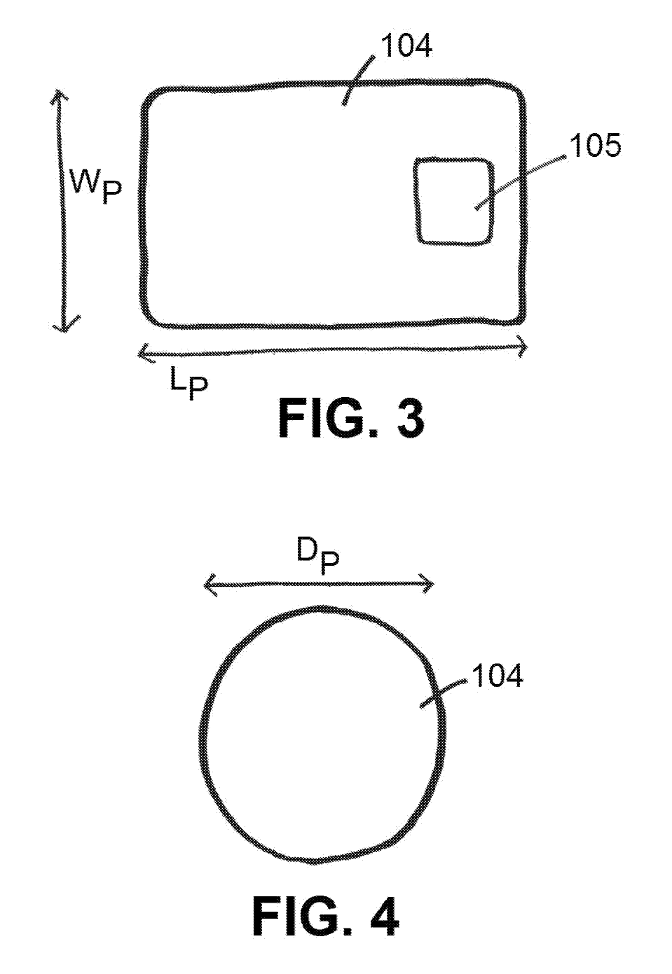

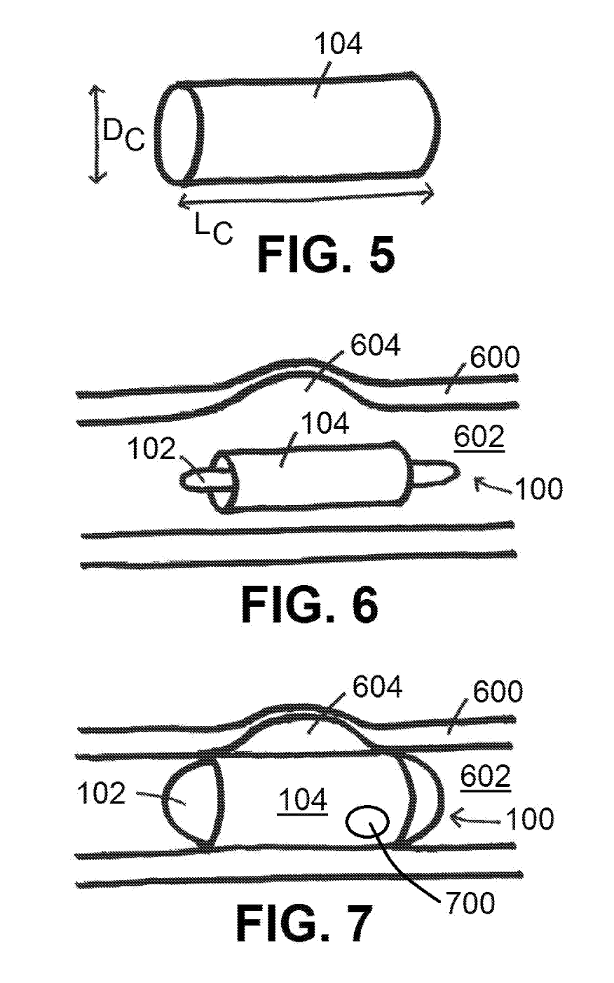

Balloons 102 can be configured having various inflated lengths (L_{B} as shown in FIG. 1) and diameters (D_{B} as shown in FIG. 1). Material layers 104, configured as patches, can also have various lengths (L_{P} as shown in FIG. 3, for example), widths (W_{P} as shown in FIG. 4, for example), and/or diameters (D_{P} as shown in FIG. 4, for example), depending on configuration of said material layers 104. Material layers, configured as cylinders, can have various lengths (L_{C} as shown in FIG. 3, for example) and diameters (D_{C} as shown in FIG. 4, for example), depending on configuration of said material layers 104. For example, for a relatively small aneurysm, a relatively small material layer 104 can be used, such as to treat a relatively small aneurysm that does not completely surround the luminal organ having the aneurysm. A material layer 104 configured as a patch or a cylinder may be used to treat such an aneurysm. For a relatively larger aneurysm, a relatively large material layer 104 can be used, such as to treat a relatively large aneurysm that completely surrounds the luminal organ having the aneurysm. In various embodiments, the dimensions (length, width, diameter, etc.) of balloon 102 can correspond to the dimensions (length, width, diameter, etc.) of material layer 104 used in connection with said balloon 102.

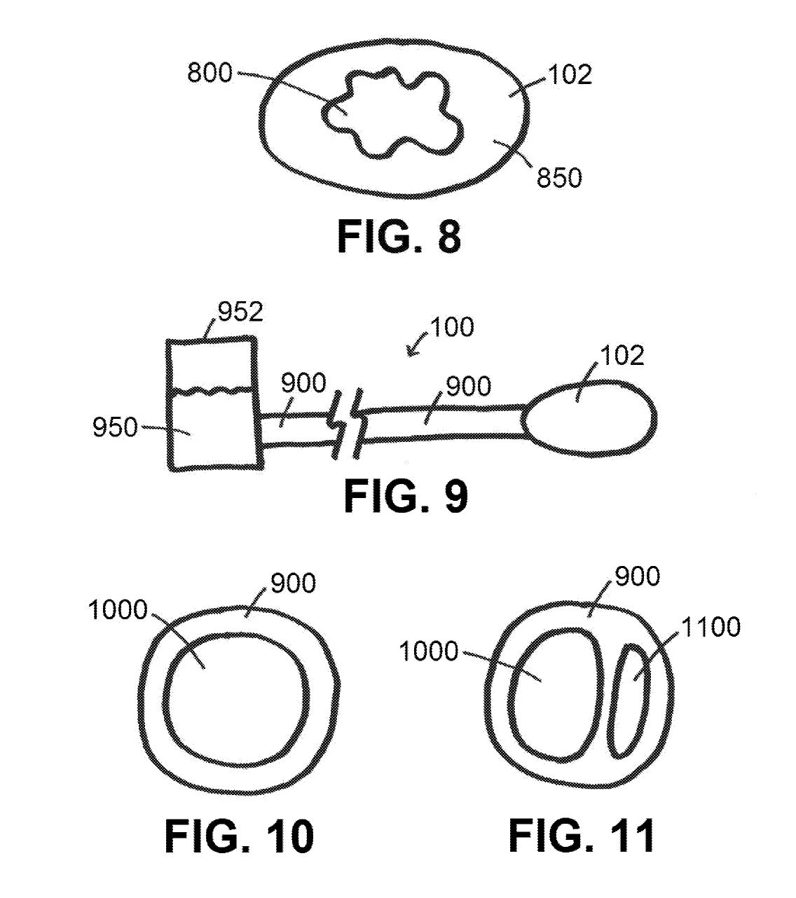

Material layers 104 of the present disclosure can be delivered into a lumen 602 of a mammalian luminal organ 600, at a location of an aneurysm 604, such as shown in FIGS. 6 and 7. Balloon 102, having a material layer 104 positioned therein (such as pre-adhered thereto, using a balloon adhesive 800 (such as shown in FIG. 8) that is either applied to an outer surface 850 of balloon 102 and/or to material layer 104 and is ultimately present between balloon 102 and material layer 104 in embodiments using said adhesive 800. Balloon 102 can be deflated or relatively deflated at the time of delivery into the lumen 602 of the mammalian luminal organ 600, such as shown in FIG. 6), and can be inflated after delivery into the lumen 602 of the mammalian luminal organ 600, such as shown in FIG. 7) so that the material layer 104 positioned upon balloon 102 contacts the mammalian luminal organ 600 at the location of the aneurysm 604, such as shown in FIG. 7.

Balloon 102, in various embodiments, can be coupled to an elongated body, such as a catheter 900 (as shown in FIG. 9), whereby a gas and/or a liquid (an inflation material 950) from an inflation source 952 (such as a syringe or other mechanism configured to deliver an inflation material 950) can be delivered therefrom, through catheter 900, such as through a first lumen 1000 of a catheter 900 having a first lumen 100 (as shown in FIG. 10) or through a first lumen 1000 or a second lumen 1100 of a catheter 900 having said lumens 1000, 1100 (as shown in FIG. 11). Said inflation material 950 can inflate balloon 900 so that material layer 104 positioned thereon can contact the mammalian luminal organ 600 at the location of the aneurysm 604, such as shown in FIG. 7. Catheter 900 having a balloon 102 coupled thereto may be referred to as a balloon catheter or an inflation catheter.

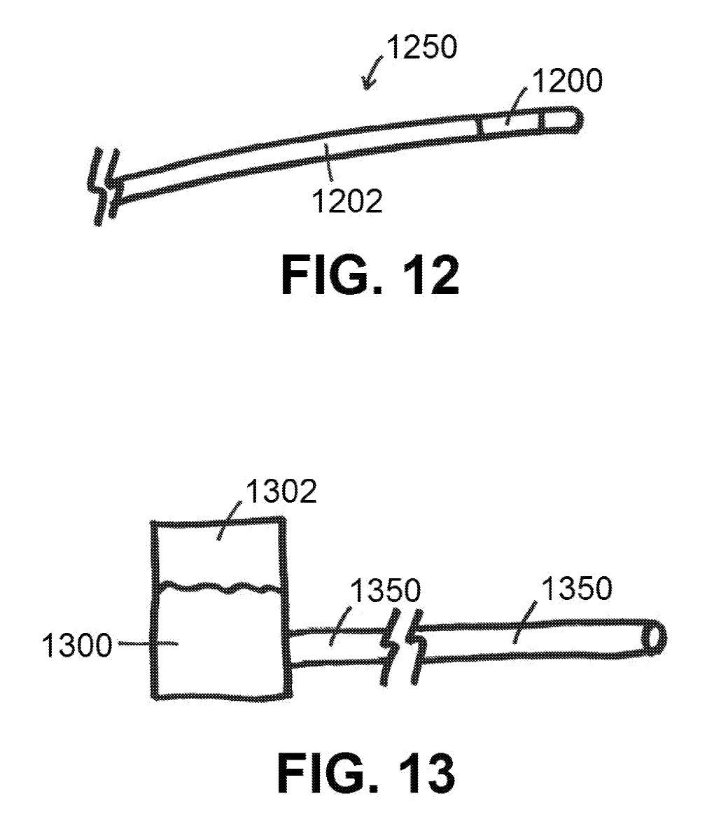

The present disclosure also includes disclosure of an ultraviolet (UV) light sensitive adhesive 700, such as shown in FIG. 7, which is positioned on a relative outside of a material layer 104 of the present disclosure, so that light sensitive adhesive 700 contacts the mammalian luminal organ 600 upon inflation of balloon 102 during delivery of material layer 104. A light source 1200, such as a UV light source 1200 (which may be a fiber optic light source 1200, in various embodiments) coupled to an elongated body 1202 (such as a catheter or a wire), such as shown in FIG. 12 and identified as a light device 1250, can be delivered into the mammalian luminal organ 600, at the location of the material layer 104 therein, and activated (turned on), so that UV light from the UV light source 1200 can activate the light sensitive adhesive 700 to cause the light sensitive adhesive 700 to cure or otherwise activate to adhere the material layer 104 to the mammalian luminal organ 600 at the location of the aneurysm 604. Such UV light could have a first wavelength or wavelength range, whereby the first wavelength (or first wavelength range) could cause the light sensitive adhesive 700 to cure or otherwise activate. Should it be desired to remove the material layer 104 from the mammalian luminal organ 600 after being adhered thereto, the UV light source 1200 could be activated once again, such as to, for example, cause a second wavelength or second wavelength range of UV light to emanate therefrom, causing the adhesive to deactivate and allow the material layer 104 to detach from the mammalian luminal organ 600.

In at least one embodiment of the present disclosure, UV light from the UV light source 1200 could be used to cure or otherwise activate the light sensitive adhesive 700, causing the material layer to attach to the mammalian luminal organ 600 at the location of the aneurysm 604. A second wavelength of UV light, such as referenced above, could be used to deactivate the light sensitive adhesive 700, or, for example, a chemical could be delivered within the mammalian luminal organ 600, whereby the chemical is configured to deactivate the light sensitive adhesive 700. Said chemical 1300 could be delivered from a chemical delivery source 1302, such as shown in FIG. 13, through a catheter 1350 coupled thereto, so that the chemical 1300 exits catheter 1350 so that chemical 1300 can contact material layer 104 and cause the light sensitive adhesive 700 to deactivate.

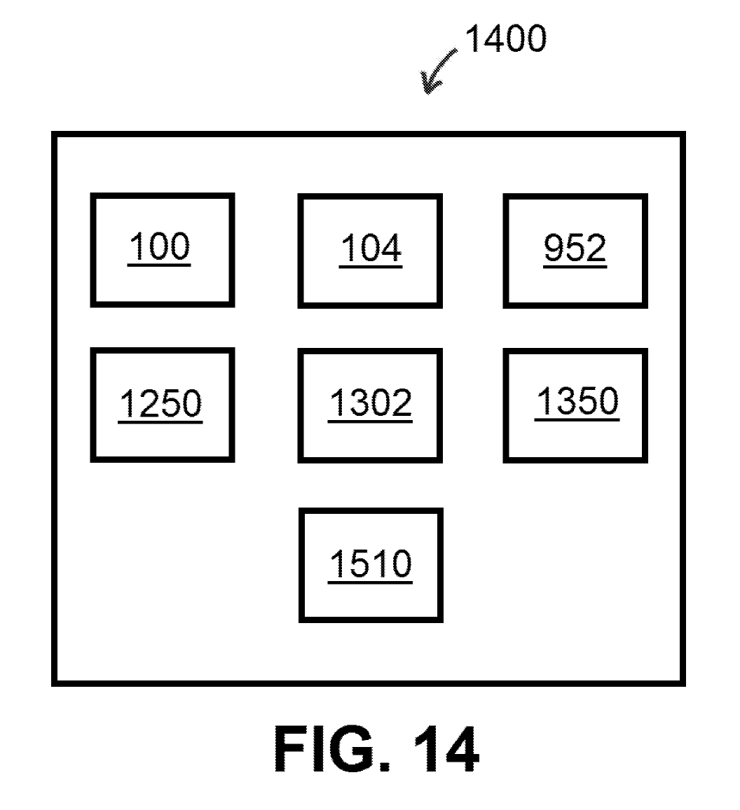

FIG. 14 shows a block diagram of components of exemplary systems 1400 of the present disclosure. As shows therein, an exemplary system 1400 of the present disclosure can comprise an exemplary device 100 of the present disclosure and one or more of a material layer 104, an inflation source 952, a light device 1250, a chemical delivery source 1302, and/or a catheter 1350, or multiples of the same, as may be desired.

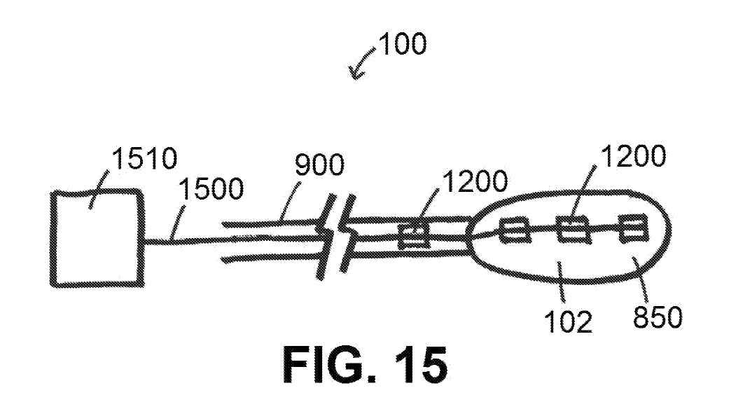

FIG. 15 shows an additional embodiment of an exemplary device 100 of the present disclosure. As shown therein, device 100 comprises a balloon 102 and further comprises one or more light sources 1200 (such as a fiber optic light source 1200) positioned within balloon 102, embedded within balloon 102, and/or positioned upon an outer surface 850 of balloon 102. For example, and in various embodiments light source(s) 102 can be coupled to a portion of catheter 900 within balloon 102, coupled to a portion of catheter 900 outside of balloon 102, embedded with the material comprising balloon 102, and/or affixed to outer surface 850 of balloon 102, configured so that when light source(s) are operated, such as to generate one or more wavelengths or wavelength ranges of UV light or other light, said light can emanate from light source(s) 1200 and be directed toward light sensitive adhesive 700 so to activate/cure said light sensitive adhesive 700 or deactivate said light sensitive adhesive 700, depending on, for example, the wavelength of light applied thereto. In embodiments where light source 1200 is coupled to catheter 900 adjacent to balloon 102, light from light source 1200 could be “seen” by the mammalian liminal organ 600 as well as a surface of material layer 104 that would be adhering to mammalian luminal organ 600. One or more wires 1500 can be used to connect to one or more light source(s) 1200, whereby said wires 1500 can be used to deliver power from a power supply 1510, such as a battery or other power source 1500, operably connected to said wires 1500.

With such an embodiment of a device 100, for example, light from light source(s) 1200 can transmit through balloon 102, such as when light source(s) 1200 are positioned within balloon 102. As light sensitive adhesive 700 would be positioned on a relative outside of material layer 104, such as shown in FIG. 1 and referenced herein, so that when balloon 102 is expanded, and material layer 104 positioned thereon contacts the mammalian luminal organ 600, the light sensitive adhesive 700 is between the material layer 104 and the mammalian luminal organ 600. When said light sensitive adhesive 700 is then activated/cured by light source(s) 1200, material layer 104 will affix/attach to mammalian luminal organ 600, such as at a location of an aneurysm 604, as may be desired. Light from light source(s) 1200 can therefore be transmitted through material layer 104 itself, so that the light sensitive adhesive 700 can be activated/cured or deactivated, as may be desired, using light from light source(s) 1200.

Procedurally, and once material layer 104 is attached to a mammalian luminal organ 600 (so to cover and treat an aneurysm 604, as generally referenced herein), device 100 will need to be removed from the mammalian luminal organ 600, as may be desired. The present disclosure includes disclosure of various mechanisms/methods to accomplish the same, including, but not limited to, mechanical approaches and/or light/chemical approaches, while allowing material layer 104 to remain affixed to the mammalian luminal organ 600. For example, and in at least one embodiment, a mechanical approach such as twisting catheter 900 (and therefore twisting balloon 102), namely rotating catheter 900 so that the bond of balloon adhesive 800 between balloon 102 and material layer 104 breaks to release balloon 102 from material layer 104, or pushing or pulling catheter 900 within mammalian luminal organ 600 so that the bond of balloon adhesive 800 between balloon 102 and material layer 104 breaks to release balloon 102 from material layer 104, whereby torsion and/or shear causes balloon 102 to disengage from material layer 104. A light/chemical approach could involve the activation of a light source 1200, such as by way of a light device 1250 or a device 100 having a light source 1200, to cause light from said light source 1200 to deactivate or otherwise weaken balloon adhesive 800 so to allow balloon 102 to disengage from material layer 104, while allowing material layer 104 to remain affixed to the mammalian luminal organ 600. With such a light approach, so long as the UV-cured adhesive force of light sensitive adhesive 700 (used to affix material layer 104 to mammalian luminal organ 600) is greater than the adhesive force of the balloon adhesive 800, said mechanical or light approaches could be successful approaches for releasing balloon 102 from material layer 104. A chemical 1300 from a chemical delivery source 1302 could also be introduced into the mammalian luminal organ 600, whereby said chemical 1300 is configured to weaken the balloon adhesive 800 so to allow balloon 102 to disengage from material layer 104. However, in various embodiments, it may be preferred not to introduce a chemical 1300 into the mammalian luminal organ 600. Regardless of approach (mechanical, light, or chemical), once balloon 102 is disengaged from material layer 104, balloon 102 can be fully or partially deflated so to allow device 100 to be removed from the mammalian luminal organ 600, such as by, for example, retracting catheter 900 coupled to balloon 102 to remove the device 100 from the mammalian luminal organ 600.

As referenced herein, various devices 100 of the present disclosure comprise a balloon 102, and use a material layer 104 to treat aneurysms, such as cerebral aneurysms or aortic aneurysms, without the use of stents, coils (such as for cerebral aneurysms) or endografts (such as for aortic aneurysms). When a material layer 104 is ultimately delivered within a mammalian luminal organ 600 and adhered thereto at a location of an aneurysm 604, pressure within the mammalian luminal organ 600 is relieved at the aneurysm 604, whereby the material layer 104 effectively covers the aneurysm 604. Should the aneurysm 604 subsequently rupture, the material layer 104 would prevent blood from escaping from the mammalian luminal organ 600 at the area of the ruptured or otherwise compromised aneurysm 604.

Various devices 100 of the present disclosure can also be applied to myocardial infarcts where device 100 can be delivered to the epicardial or endocardial surface of the heart. Various devices 100 of the present disclosure can also have a drug release portion 105 comprising a drug to be released over time, such as, for example, one or more of anti-inflammatory agents, growth agents to regenerate vessel smooth muscle or cardiac muscle, and the like. Various devices 100 of the present disclosure can also be configured to be bio-resorbable using known Bioresorbable materials (metals or polymers) to provide short term (6 or 9 months) therapy, for example.

While various embodiments of devices, systems, and methods for treating aneurysms and other medical conditions have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A device, comprising:
an expandable member; and
a material layer coupled to the expandable member;
wherein the expandable member is configured to attach to an elongated body configured to deliver an inflation material therethrough and into the expandable member.

2. The device of claim 1, wherein the material layer is pre-adhered to the expandable member using an adhesive.

3. The device of claim 1, wherein the material layer does not completely encircle a portion of the expandable member.

4. The device of claim 1, wherein the expandable member comprises a mammalian tissue selected from the group consisting of pulmonary ligament, parietal pleura, mediastinal pleura, and visceral pleura.

5. The device of claim 1, wherein the expandable member comprises a non-mammalian tissue.

6. The device of claim 1, wherein the expandable member is not configured as a stent, a coil, an endograft, or an endoprosthesis.

7. The device of claim 1, wherein the material layer is configured as a patch.

8. The device of claim 1, wherein the material layer is configured as a cylinder.

9. The device of claim 1, further comprising an ultraviolet light sensitive adhesive positioned on a relative outside of the material layer, the ultraviolet light sensitive adhesive configured to cure or otherwise activate from ultraviolet light so to cause the ultraviolet light sensitive adhesive to adhere the material layer to a mammalian luminal organ when the device is positioned within a lumen of the mammalian luminal organ and when the expandable member is expanded.

10. The device of claim 1, further comprising an ultraviolet light sensitive adhesive positioned on a relative outside of the material layer, the ultraviolet light sensitive adhesive configured to cure or otherwise activate from a first wavelength of ultraviolet light so to cause the ultraviolet light sensitive adhesive to adhere the material layer to a mammalian luminal organ when the device is positioned within a lumen of the mammalian luminal organ and when the expandable member is expanded, and wherein the ultraviolet light sensitive adhesive is further configured to deactivate from a second wavelength of ultraviolet light so to cause the ultraviolet light sensitive adhesive to detach the material layer from the mammalian luminal organ.

11. The device of claim 1, further comprising one or more light sources positioned within the expandable member.

12. The device of claim 1, further comprising one or more light sources positioned upon an outer surface of the expandable member.

13. The device of claim 1, further comprising a drug release portion positioned upon the material layer, so that when the expandable member is inflated within a lumen of a mammalian luminal organ, the drug release portion contact the mammalian luminal organ to allow drug from the drug release portion to be delivered to the mammalian luminal organ.

14. A system, comprising:
a device, comprising:
an expandable member; and
a material layer coupled to the expandable member;
an elongated body coupled to the expandable member; and
a chemical delivery source configured to couple to the elongated body;
wherein the elongated body is configured to deliver an inflation material from the chemical delivery source therethrough and into the expandable member.

15. The system of claim 14, further comprising a light device configured for insertion into a lumen of a mammalian luminal organ with the device, the light device further configured to generate ultraviolet light within the lumen.

16. A method, comprising the steps of:
introducing a device into a lumen of a mammalian luminal organ at or near an aneurysm, the device comprising:
an expandable member; and
a material layer coupled to the expandable member;
wherein the expandable member is configured to attach to an elongated body configured to deliver an inflation material therethrough and into the expandable member; and
inflating the expandable member so that the material layer contacts the mammalian luminal organ and adheres to the mammalian luminal organ at or near the aneurysm.

17. The method of claim 16, further comprising the step of:
deflating the expandable member so to cause the expandable member to detach from the material layer.

18. The method of claim 16, wherein the material layer has an adhesive positioned thereon, and wherein the step of inflating is performed to cause the adhesive of the material layer to contact the mammalian luminal organ and to cause the material layer to adhere to the mammalian luminal organ.

19. The method of claim 16, further comprising the step of:
Introducing a chemical into the expandable member to cause an adhesive applied to the expandable member to weaken to allow the expandable member to disengage from the material layer.

20. The method of claim 16, wherein the step of inflating is performed to cause the material layer to adhere to the mammalian luminal organ and to completely cover the aneurysm.
